(19) United States
(12) Patent Application Publication
(10) Pub. No.: US 2009/0143457 A1
(43) Pub. Date: Jun. 4, 2009

(54) POLYCYCLIC COMPOUNDS FOR USE IN TREATING OCULAR NEURODEGENERATIVE DISEASES

(75) Inventors: Cornelis J. Van der Schyf, Akron, OH (US); Randolph B. Schiffer, Lubbock, TX (US); Paula Grammas, Lubbock, TX (US); Masao Roy Wilson, Denver, CO (US); Werner J. Geldenhuys, Kent, OH (US); Lois-May Bezuidenhout, Rootstown, OH (US)

Correspondence Address: WALKER & JOCKE, L.P.A., 231 SOUTH BROADWAY STREET, MEDINA, OH, 44256, US

(73) Assignee: NORTHEASTERN OHIO UNIVERSITIES COLLEGE OF MEDICINE, Rootstown, OH (US)

(21) Appl. No.: 12/328,238

(22) Filed: Dec. 4, 2008

Related U.S. Application Data
(60) Provisional application No. 60/992,236, filed on Dec. 4, 2007.

(51) Int. Cl. C07D 209/56; A61K 31/403; A61P 27/02

(52) U.S. Cl. 514/410; 548/420

(57) ABSTRACT

Described herein are various compounds for treatment of ocular neurodegenerative diseases, including but not limited to glaucoma and diabetic retinopathy. The compounds described herein can act to attenuate and/or block calcium release from external neuronal environments as well as intracellular stores.

### CROSS REFERENCE TO RELATED APPLICATION

This application claims the benefit of U.S. Provisional Patent No. 60/992,236, filed on Dec. 4, 2007, and entitled “POLYCYCLIC COMPOUNDS FOR USE IN TREATING GLAUCOMA”, the entirety of which is incorporated herein by reference.

### BACKGROUND

Glaucoma and diabetic retinopathy are complex diseases with numerous risk factors and mechanisms that can ultimately lead to ganglion cell death and blindness. With respect to glaucoma, such term refers to a group of eye diseases that can gradually cause an individual to lose their sight. More specifically, glaucoma is a relatively common retinal disease characterized by progressive neurodegenerative death of retinal ganglion cells (RGCs) (e.g., output neurons of the retina). This disease can lead to slowly progressive vision loss and, eventually, blindness.

Diabetic retinopathy refers to a disease that causes damage to the retina of an eye caused by complications corresponding to diabetes. Diabetic retinopathy is an ocular manifestation of a systemic disease which has been shown to affect approximately eighty percent of individuals that have had diabetes for ten years or more. Diabetic retinopathy, however, often has few or no early warning signs, and the particular trigger for diabetic retinopathy has been unknown.

Referring again to glaucoma, two major categories of glaucoma are hypertensive glaucoma and normotensive glaucoma. The underlying neuropathology of glaucoma is still under investigation, but studies have indicated that ischemic events brought on by an initial trigger of high intraocular pressure in the case of hypertensive glaucoma, and vascular abnormality in the case of normative glaucoma, lead to progressive death of RGCs in both types of glaucoma. In some cases, however, a person found to have high intraocular pressure will not become afflicted with glaucoma while in other cases a person found to have intraocular pressure that is within a “normal” range will become afflicted with glaucoma. Thus, while increased intraocular pressure may be a cause of glaucoma in some cases, in other cases it may be an underlying cause for a different problem.

### SUMMARY

The following is a brief summary of subject matter that is described in greater detail herein. This summary is not intended to be limiting as to the scope of the claims.

Compounds, apparatuses, and methodologies pertaining to treatment of ocular neurodegenerative diseases are described in detail below. For instance, a polycyclic compound can be used to combat neural death as found in an eye that suffers from an ocular neurodegenerative disease, such as glaucoma or diabetic retinopathy.

The polycyclic compounds described herein can perform multiple mechanisms of action on the eye, both in attenuating excitotoxicity by modulating/attenuating calcium entry through the NMDA receptor channel and acting as an L-type calcium channel blocker. The NMDA receptor channel and L-type calcium channel can be found in ocular neurons within the retina of the eye as well as neurons in the optic nerve, for example.

The polycyclic compounds described herein may also act to block/attenuate calcium release from intracellular stores. For example, the compounds described herein may act to block/attenuate calcium release by way of the endoplasmic reticulum and/or the mitochondria.

Other aspects will be appreciated upon reading and understanding the attached figures and description.

### BRIEF DESCRIPTION OF THE DRAWINGS

FIGS. 1a-1l are example compounds that can be used in connection with treating ocular neurodegenerative diseases.

FIG. 2 is an example graph that illustrates data pertaining to NMDA channel block.

FIG. 3 is an example graph that illustrates that at least a subset of the compounds described above have an affinity for the MK-801 binding site in the NMDA channel.

FIG. 4 is an example graph that illustrates a dose-dependent effect of an example compound on [³H]MK-801 binding in the presence of NMDA (100 μM) and Gly (100 μM).

FIG. 5 is an illustration of a container that includes an aqueous solution comprising a polycyclic compound.

FIG. 6 is an illustration of an application of an aqueous solution that includes a polycyclic compound to an eye suffering from a neurodegenerative disease.

FIG. 7 is flow diagram illustrating a methodology for preparing an aqueous solution including a polycyclic compound for treatment of ocular neurodegenerative diseases.

FIG. 8 is a flow diagram illustrating a methodology for prescribing treatment to a sufferer of an ocular neurodegenerative disease.

FIG. 9 is a flow diagram illustrating a methodology for applying an aqueous solution to an eye that suffers from an ocular neurodegenerative disease.

### DETAILED DESCRIPTION

Various compounds, apparatuses, and methodologies are described herein pertaining to treatment of neurodegenerative diseases of the eye in general, and treatment of glaucoma and/or diabetic retinopathy in particular.

Glaucoma and diabetic retinopathy are complex diseases with numerous risk factors and mechanisms that ultimately lead to ganglion cell death and blindness. At least most of the Food and Drug Administration (FDA)-approved glaucoma medications are directed toward lowering intraocular pressure. The inventors of the compounds, apparatuses, and methodologies described herein, however, are aware that pressure-independent disease mechanisms can lead to development of glaucomatous optic neuropathy, including excitotoxicity, a glutamate and calcium-dependent process.

Regardless of an initial trigger for glaucoma or diabetic retinopathy, the inventors are aware that abnormally high levels of cytosolic free calcium (Ca²⁺) constitutes an early trigger in a cascade of events that lead to neuronal damage under pathological conditions in many neurodegenerative diseases, including glaucoma and diabetic retinopathy. High intracellular free Ca2+ can cause activation of various enzymes and death proteins, followed by mitochondrial and cell membrane injury. Such responses can mediate the cytotoxicity that eventually leads to neuronal death, also in the case of RGCs in glaucoma. Thus, alterations of Ca²⁺ transporting proteins in a plasma membrane (ligand and voltage-gated Ca²⁺ channels, ion-motive ATPases, glutamate receptors), endoplasmic reticulum (ryanodine and inositol triphosphate receptors), and mitochondria (electron transport chain proteins, Bcl-2 family members, and uncoupling proteins) can be implicated in neuronal (and therefore, retinal) dysfunction and disease. As noted above, diabetic retinopathy is also a chronic degenerative retinal disease that leads to progressive vision loss and/or blindness. Similar events that lead to the pathology of glaucoma, including ischemia and glutamate excitotoxicity with resulting cytosolic Ca²⁻ overload (both from an extracellular environment and from intracellular stores in the eye) can contribute to RGC injury in diabetic retinopathy. Thus, the compounds described herein can be used for treating glaucoma and also for treating diabetic retinopathy, amongst other ocular neurodegenerative diseases.

Furthermore, the inventors of the compounds, apparatuses, and methodologies described herein recognize that glutamate ligand-activated calcium channels [or N-methyl D-aspartate (NMDA) channels] and L-type calcium channels are desirable targets for a compound/compounds that act to block/attenuate calcium from entering neurons in the eye.

Accordingly, described herein are various compounds (e.g., polycyclic compounds) that can be used in connection with treating glaucoma and/or diabetic retinopathy amongst other ocular neurodegenerative diseases. At least one of these compounds can, for instance, be placed in an aqueous solution that is configured for topical application to a surface of the eyeball. In another example, at least one of the compounds shown below can be placed in a dosage form for oral consumption. In yet another example, at least one of the compounds described herein can be placed in a semi-aqueous substance for application on the skin near the eyeball. In still yet another example, at least one of the compounds described herein can be placed in an aqueous solution that is configured for injection to the eye via a needle. One skilled in the art will recognize and appreciate other delivery mechanisms for treatment of glaucoma, diabetic retinopathy, and other ocular neurodegenerative diseases, and such mechanisms are contemplated and intended to fall under the scope of the hereto-appended claims.

More particularly, shown below are example azatricyclo[6.3.0.0^{2,6}] compounds that can have the following general formula:

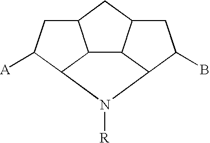

where R can be a linear or branched alkyl group that has one to twelve carbon atoms. R may also include a hydroxyl or halogen substituent. In another example, R can be a phenyl group. In yet another example, R can be a phenyl group that is substituted with a linear, cyclic or branched alkyl group having one to twelve carbon atoms. The alkyl group may also optionally include a hydroxyl or halogen substituent.

Pursuant to numerous examples, R can be any one of —CH₃, —(CH₂)₃—CH₃, —(CH₂)₇—CH₃, —CH₂—C₆H₅, —(CH₂)₂—C₆H₅, —(CH₂)₃, C₆H₅, —CH₂CH₃, —(CH₂)₁₁—CH₃, —CH₂CH(CH₃)₃, —(CH₂)₅—CH₃, —C(CH₃)₂—CH₂—C(CH₃)₃, —(CH₂)₉—CH₃, —CH₂—CH₂—OH, or —C₆H₅, amongst others.

In addition, A and/or B can be hydrogen. In another example, at least one of A or B can be a methyl group. Furthermore, the above-illustrated compound can be combined with a diluent, and the combination can be administered to a patient and/or prescribed to a patient. The amount of the compound in the combination can be an amount effective to be used as an L-type calcium antagonist, an NMDA receptor antagonist, an inositol-1,4,5-trisphosphate receptor channel (InsP3R) antagonist, and/or a ryanodine receptor channel antagonist. The above compound in any of its forms can be used to treat glaucoma and/or diabetic retinopathy (e.g., as an agent against glaucomatous retinal neurodegeneration and/or diabetic retinopathy), amongst other ocular neurodegenerative diseases.

Also shown below are example 4-azahexacyclo[5.4.1.0^{2,6}.0.0^{5,9}.0^{8,11}]dodecan compounds of the following general formula:

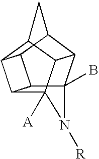

where R can be a linear or branched alkyl group that has one to twelve carbon atoms. R may also include a hydroxyl or halogen substituent. In another example, R can be a phenyl group. In yet another example, R can be a phenyl group that is substituted with a linear, cyclic or branched alkyl group having one to twelve carbon atoms. The alkyl group may also optionally include a hydroxyl or halogen substituent.

Pursuant to numerous examples, R can be any one of —CH₃, —(CH₂)₃—CH₃, —(CH₂)₇—CH₃, —CH₂—C₆H₅, —(CH₂)₂—C₆H₅, —(CH₂)₃—C₆H₅, —CH₂CH₃, —(CH₂)₁₁—CH₃, —CH₂CH(CH₃)₃, —(CH₂)₅—CH₃, —C(CH₃)₂—CH₂—C(CH₃)₃, —(CH₂)₉—CH₃, —CH₂—CH₂—OH, or —C₆H₅, amongst others.

In addition, A and/or B can be hydrogen. In another example, A or B can be a hydroxyl group. In yet another example, at least one of A or B can be a methyl group. Furthermore, the above-illustrated compound can be combined with a diluent, and the combination can be administered to a patient and/or prescribed to a patient. The amount of the compound in the combination can be an amount effective to be used as an L-type calcium antagonist, an NMDA receptor antagonist, an inositol-1,4,5-trisphosphate receptor channel (InsP3R) antagonist, and/or a ryanodine receptor channel antagonist. The above compound in any of its forms can be used to treat glaucoma and/or diabetic retinopathy (e.g., as an agent against glaucomatous retinal neurodegeneration and/or diabetic retinopathy) or other ocular neurodegenerative disease.

In addition, shown below are 8-Substituted-8,11-oxapentacyclo[5.4.0.0^{2,6}.0^{3,10}.0^{5,9}]undecane compounds of the following general formula:

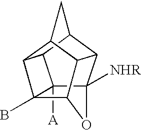

where R can be a linear or branched alkyl group that has one to twelve carbon atoms. R may also include a hydroxyl or halogen substituent. In another example, R can be a phenyl group. In yet another example, R can be a phenyl group that is substituted with a linear, cyclic or branched alkyl group having one to twelve carbon atoms. The alkyl group may also optionally include a hydroxyl or halogen substituent.

Pursuant to numerous examples, R can be any one of —CH₃, —(CH₂)₃—CH₃, —(CH₂)₇—CH₃, —CH₂—C₆H₅, —(CH₂)₂—C₆H₅, —(CH₂)₃—C₆H₅, —CH₂CH₃, —(CH₂)₁₁—CH₃, —CH₂CH(CH₃)₃, —(CH₂)₅—CH₃, —C(CH₃)₂—CH₂—C(CH₃)₃, —(CH₂)₉—CH₃, —CH₂—CH₂—OH, or —C₆ H₅, amongst others.

In addition, A and/or B can be hydrogen. In another example, at least one of A or B can be a methyl group. Furthermore, the above-illustrated compound can be combined with a diluent, and the combination can be administered to a patient and/or prescribed to a patient. The amount of the compound in the combination can be an amount effective to be used as an L-type calcium antagonist, an NMDA receptor antagonist, an inositol-1,4,5-trisphosphate receptor channel (InsP3R) antagonist, and/or a ryanodine receptor channel antagonist. The above compound in any of its forms can be used to treat glaucoma and/or diabetic retinopathy (e.g., as an agent against glaucomatous retinal neurodegeneration and/or diabetic retinopathy), or other ocular degenerative diseases.

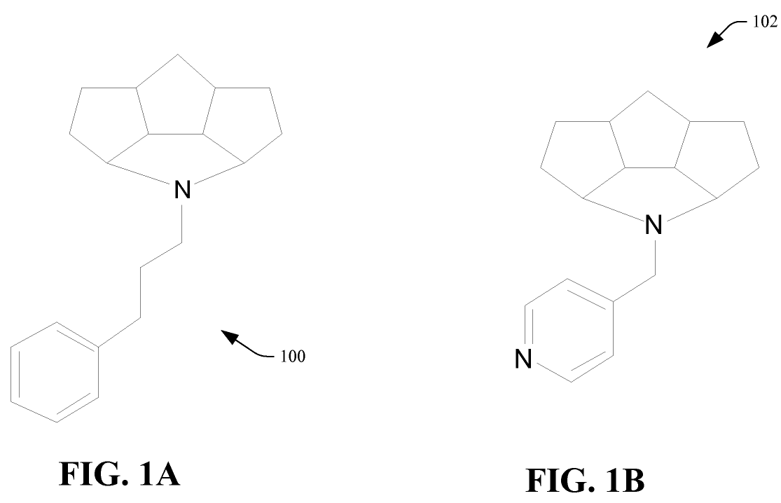

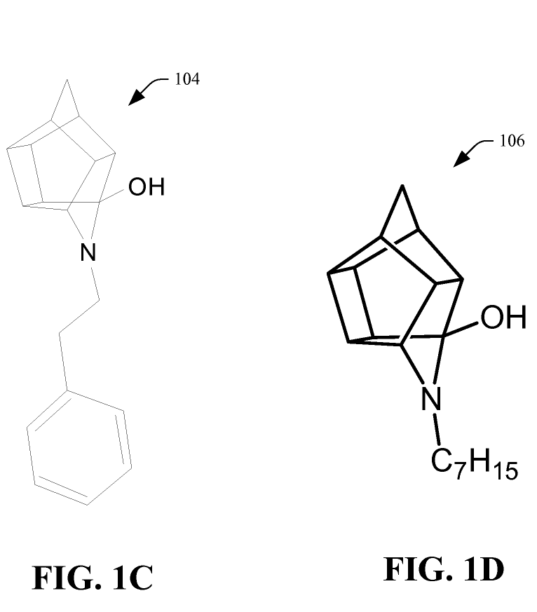

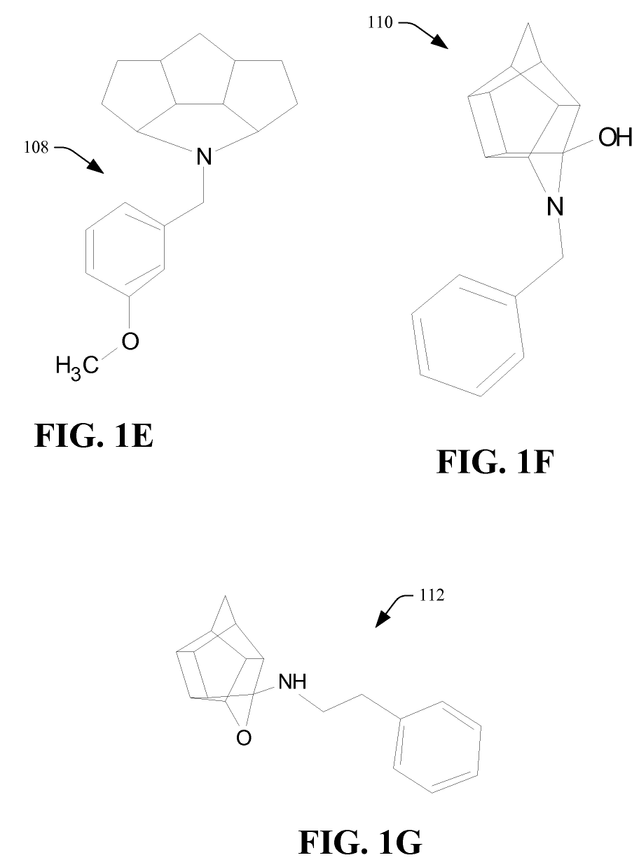

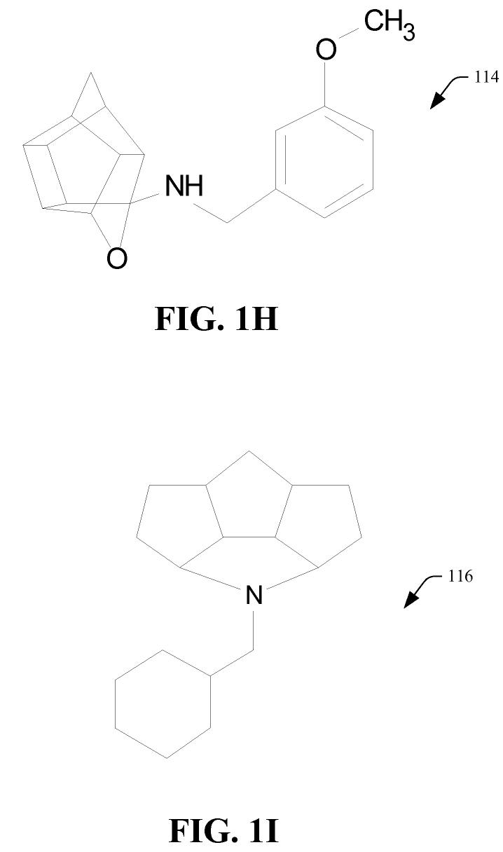

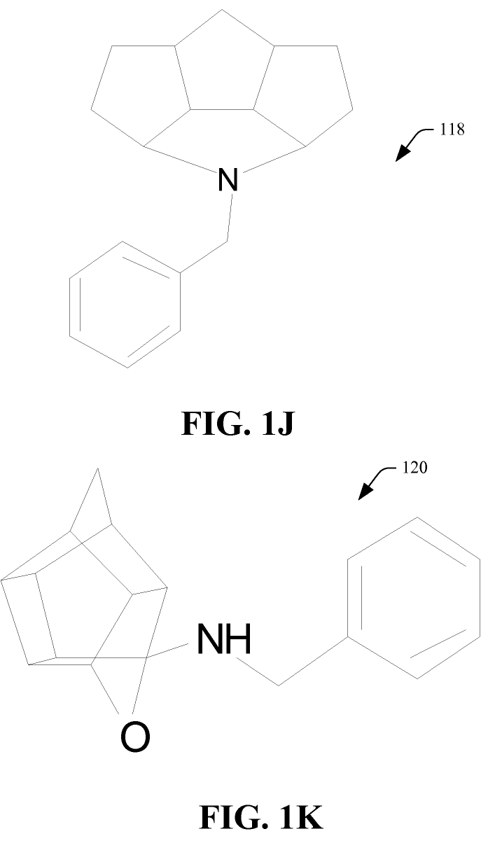

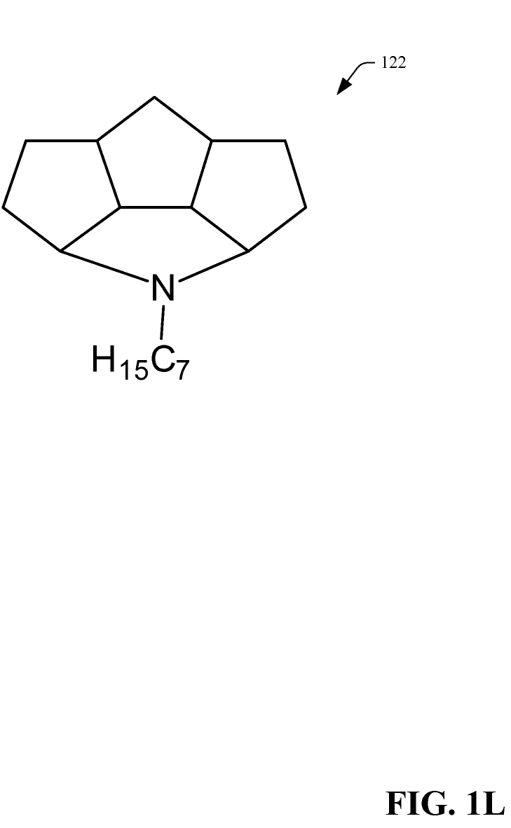

Example compounds that conform to at least one of the general structures described above are now described in detail. Referring now to FIG. 1a, a first example compound 100 that can be used in connection with treating glaucoma, diabetic retinopathy, or other suitable ocular neurodegenerative disease is illustrated. The compound 100 has an IUPAC name of N-phenylpropyl-3,11-azatricyclo[6.3.0.0^{2,6}]undecane. The compound 100 has an IC₅₀ value with respect to the L-type calcium channel of 1.4 μM.

FIG. 1b illustrates a second example compound 102 that can be used in connection with treating glaucoma and/or diabetic retinopathy, or other suitable ocular neurodegenerative disease. The compound 102 has an IPUAC name of N-(pyridin-4-ylmethyl)-3,11-azatricyclo[6.3.0.0^{2,6}]undecane. The compound 102 has an IC₅₀ value with respect to the L-type calcium channel of 4.9 μM.

FIG. 1c illustrates a third example compound 104 that can be used in connection with treating glaucoma and/or diabetic retinopathy, or other suitable ocular neurodegenerative disease. The compound 104 has an IPUAC name of N-phenylethyl-4-azahexacyclo[5.4.1.0^{2,6}.0.0^{5,9}.0^{8,11}]dodecan-3-ol. The compound 104 has an IC₅₀ value with respect to the L-type calcium channel of 6.8 μM.

Referring now to FIG. 1d, a fourth example compound 106 that can be used in connection with treating glaucoma and/or diabetic retinopathy, or other suitable ocular neurodegenerative disease is illustrated. The compound 104 has an IPUAC name of N-heptyl-4-azahexacyclo[5.4.1.0^{2,6}.0.0^{5,9}.0^{8,11}]dodecan-3-ol. The compound 106 has an IC₅₀ value with respect to the L-type calcium channel of 7.5 μM.

FIG. 1e illustrates a fifth example compound 108 that can be used in connection with treating glaucoma and/or diabetic retinopathy, or other suitable ocular neurodegenerative disease. The compound 108 has an IPUAC name of N-(3-methoxybenzyl)-3,11-azatricyclo[6.3.0.0^{2,6}]undecane. The compound 108 has an IC₅₀ value with respect to the L-type calcium channel of 11 μM.

With reference now to FIG. 1f, a sixth example compound 110 is illustrated, wherein the compound 110 can be used in connection with treating glaucoma and/or diabetic retinopathy or other suitable ocular neurodegenerative disease. The compound 110 has an IPUAC name of N-benzyl-4-azahexacyclo[5.4.1.0^{2,6}.0.0^{5,9}.0^{8,11}]dodecan-3-ol. The compound 110 has an IC₅₀ value with respect to the L-type calcium channel of 32.9 μM.

FIG. 1g illustrates a seventh example compound 112 that can be used in connection with treating glaucoma and/or diabetic retinopathy or other suitable ocular neurodegenerative disease. The compound 110 has an IPUAC name of 8-phenylethylamino-8,11-oxapentacyclo[5.4.0.0^{2,6}.0^{3,10}.0^{5,9}]undecane. The compound 112 has an IC₅₀ value with respect to the L-type calcium channel of 36.4 μM.

Referring not to FIG. 1h, an eighth example compound 114 that can be used in connection with treating glaucoma and/or diabetic retinopathy or other suitable ocular neurodegenerative disease is illustrated. The compound 114 has an IPUAC name of 8-(3-methoxybenzylamino)-8,11-oxapentacyclo[5.4.0.0^{2,6}.0^{3,10}.0^{5,9}]undecane. The compound 112 has an IC₅₀ value with respect to the L-type calcium channel of 36.9 μM.

FIG. 1i illustrates a ninth example compound 116 that can be used in connection with treating glaucoma and/or diabetic retinopathy or other suitable ocular neurodegenerative disease. The compound 116 has an IPUAC name of N-cyclohexylmethyl-3,11-azatricyclo[6.3.0.0^{2,6}]undecane. The compound 116 has an IC₅₀ value with respect to the L-type calcium channel of 46 μM.

FIG. 1j illustrates a tenth example compound 118 that can be used in connection with treating glaucoma and/or diabetic retinopathy or other suitable ocular neurodegenerative disease. The compound 118 has an IPUAC name of N-benzyl-3,11-azatricyclo[6.3.0.0^{2,6}]undecane. The compound 116 has an IC₅₀ value with respect to the L-type calcium channel of 53 μM.

With reference now to FIG. 1k, a twelfth example compound 120 that can be used in connection with treating glaucoma and/or diabetic retinopathy or other suitable ocular neurodegenerative disease is illustrated. The compound 120 has an IPUAC name of 8-benzylamino-8,11-oxapentacyclo[5.4.0.0^{2,6}.0^{3,10}.0^{5,9}]undecane, and has been referred to as NGP1-01. The compound 120 has an IC₅₀ value with respect to the L-type calcium channel of 60 μM.

FIG. 1l illustrates an eleventh example compound 122 that can be used in connection with treating glaucoma and/or diabetic retinopathy or other suitable ocular neurodegenerative disease. The compound 122 has an IPUAC name of N-heptyl-3,1-azatricyclo[6.3.0.0^{2,6}]undecane. The compound 122 has an IC₅₀ value with respect to the L-type calcium channel of 55 μM.

The compounds of the general structures shown above (including those shown in FIGS. 1a-1l) can be multimodal (multimechanistic) in nature. More particularly, traditionally, clinicians treat patients by combining drugs with different therapeutic mechanisms, an approach termed polypharmacology. Often, this combination of drugs is administered in the form of two or more individual dosage forms. To simplify dosing regimens and improve patient compliance, multi-component drugs have become more popular. Multi-component drugs are drugs that include two or more agents, wherein the two or more agents are co-formulated in a single dosage form. The polycyclic components shown and described above are chemical entities that are multimodal individually. That is, the components can modulate multiple drug targets substantially simultaneously with respect to treatment of a disease.

As will be described in greater detail below, the compounds of the general structures above (including those shown in FIGS. 1a-1l) can be multimodal in that such compounds can act as a designed multiple ligand both for the L-type calcium channel and for the ligand-operated glutamatergic NMDA channel. In addition, the compounds of the general structure above can attenuate Ca²⁺ release from intracellular stores, such as the endoplasmic reticulum (ER) and mitochondria, through the inositol-1,4,5, trisphosphate receptor (InsP3R) and ryanodine receptor. Moreover, one or more of the compounds of the general structures shown above may act as a moderate sigma receptor agonist.

The compounds of the general structures shown above acting as multimodal compounds will now be described. As previously indicated herein, the inventors have determined that abnormally elevated levels of Ca²⁺ constitutes an early event in the cascade of events that lead to neuronal damage under pathological conditions in many neurodegenerative diseases. High intracellular free Ca²⁺ can cause activation of various enzymes and death proteins, followed by mitochondrial and cell membrane injury. Such activation can mediate the cytotoxicity that eventually leads to neuronal death (also in the case of RGCs in glaucoma). Thus, alterations of Ca²⁺ transporting proteins in the plasma membrane (ligand-gated and voltage-gated Ca²⁺ channels, ion-motive ATPases, glutamate receptors), endoplasmic reticulum (ryanodine and inositol triphosphate receptors), and mitochondria (electron transport chain proteins, Bcl-2 family members, and uncoupling proteins) are implicated in neuronal (and therefore, retinal) dysfunction and disease. Diabetic retinopathy, similar to glaucoma, also is a chronic degenerative disease that leads to progressive vision loss and blindness. Similar events that lead to the pathology of glaucoma, including ischemia and glutamate excitotoxicity with resulting cytosolic Ca²⁺ overload—both from the extracellular environment and from intracellular stores—also contribute to RGC injury in diabetic retinopathy.

Two major sources of calcium operate through a number of mechanisms to cause elevation of cytosolic free Ca²⁺. First, Ca²⁺ influx from the extracellular environment through calcium and non-selective cation channels imbedded in the cell membrane can cause elevation of cytosolic free Ca²⁺. Second, Ca²⁺ release from intracellular stores, exemplified first by the endoplasmic reticulum (ER) and, to a lesser extent, by the mitochondria, through specialized channel receptor complexes, such as the inositol-1,4,5-trisphosphate receptor channels (InsP3R), and from the extracellular compartment through ion channels (L-type voltage operated calcium channels, NMDA receptors) on the cell membrane can trigger additional release from intracellular stores through the ryanodine receptor through a calcium-induced calcium release mechanism, or by activating InsP through a second messenger mechanism. These mechanisms have been demonstrated to operate in concert.

RGC neurodegeneration and Ca²⁺ influx from the extracellular environment to the cytosol will now be described. With respect to NMDA receptors, glutamate, together with its co-agonist glycine, is a major excitatory neurotransmitter in the brain, including RGCs. The biological actions of glutamate are mediated by a variety of receptors, including the NMDA receptor, an ionotropic receptor coupled with a non-selective cation channel that has a high affinity for Ca²⁺. Glutamate-induced neurotoxicity, also called excitotoxicity, is mediated by Ca²⁺ overload that occurs through the NMDA receptor due to its high Ca²⁺ permeability. Calcium entering the neuron through NMDA channels can stimulate more calcium release from intracellular stores, as discussed above, to amplify cellular toxicity. With respect to L-type calcium channels, several L-type calcium channel blockers can reduce voltage-mediated and NMDA-stimulated Ca²⁺ influx in a dose-related fashion. Of the voltage-dependent Ca²⁺ channels, L-type channels can contribute up to 50% of the NMDA-stimulated influx of Ca²⁺ into the mammalian retina.

RGC neurodegeneration and endoplasmic reticular calcium dyshomeostasis (Ca²⁺ release from intracellular stores) will now be described herein. The ER is an organelle involved in neuronal signaling, including signaling in RGCs. The ER serves as a dynamic Ca²⁻ depot to facilitate rapid signaling associated with cell stimulation through electrical (action potential) or chemical (neurotransmitter) signals. This function is supported, amongst other mechanisms, by the Ca²⁺ release receptor (channels) including inositol-1,4,5-trisphosphate receptors (InsP3), and ryanodine receptors, located in the ER membrane. Disruption of intra-ER calcium homeostasis triggers an array of cellular stress responses and is intimately involved in neurodegeneration through apoptotic and neuronal cell death mechanisms.

InsP3Rs are ligand-gated intracellular Ca²⁺ channels that mediate release of Ca²⁺ from intracellular stores into the cytosol upon activation by a second messenger inositol-1,4,5-trisphosphate (InsP3). The InsP3 receptor interacts with other signaling mechanisms—such as voltage and ligand operated calcium channels—that control levels of cytosolic Ca²⁺, suggesting that the maintenance of Ca²⁺ homeostasis in normal cells are controlled by the activity of the InsP3R. In mammalian RGCs, InsP3 receptor isoforms are localized intracellularly on the ER membranes with isoform Types 1 and 3 located through the cell, and Type 2 predominantly in soma. InsP3Rs mediate changes in cytosolic Ca²⁺ concentrations that control synaptic transmission, differentiation, and apoptotic cell death. As such, InsP3R-generated cytosolic Ca²⁺ dyshomeostasis will control RGC pathophysiology in cases of neurodegenerative insult such as found in glaucoma and diabetic retinopathy.

With respect to ryanodine receptors and retinal neurodegeneration, Dantrolene, a ryanodine receptor antagonist, has been used clinically to treat malignant hyperthermia and muscle spasticity. Such drug acts by inhibiting Ca²⁺ release from ER stores by way of the ryanodine receptor channel. In neuronal cells, Dantrolene has been shown to inhibit both elevation of cytosolic Ca²⁺ levels and the neurotoxicity evoked by NMDA, glutamate, and potassium depolarization. Similar to InsP3R, ryanodine-generated cytosolic Ca²⁺ dyshomeostasis will exacerbate or cause RGC pathophysiology in cases of neurodegenerative insult following glaucomatous conditions or diabetic retinopathy.

Moreover, the compounds of the general structures shown above can be capable of crossing the Blood-Brain Barrier (BBB), the Blood-Corneal/Scleral Barrier (BSB), and/or the Blood-Retinal Barrier (BRB). Thus, compounds conforming to one of these general structures may be administered topically, orally, and/or intravenously. It is also possible that at least one compound that conforms to one of the general structures shown above will fail to cross the BBB, the BSB, and/or the BRB. Accordingly, the at least one compound may be administered via intraocular injection to the retina. Still further, a compound conforming to one of the general structures shown above may be administered by way of systematic dosing or intra or periocular injection.

It may be desirable, however, to topically administer one or more compounds that conform to the above general structures. Accordingly, solubility and permeability through biological barriers can be increased through a variety of options. Cyclodextrins are cyclic oligomers of glucose molecules. β-Cyclodextrin includes seven sugars in its ring molecule, and cyclodextrins are able to form host-guest complexes with hydrophobic molecules, thereby hosting such molecules in the interior of the toroid structure. The interior of the torus may be less hydrophilic when compared to the aqueous environment (solvent) and thus able to accommodate a lipophilic compound (and essentially hide the lipophilic compound from the hydrophilic environment). The exterior of the torus is sufficiently hydrophilic to afford cyclodextrin complexes substantial water solubility. Inclusion complexes of cyclodextrins with hydrophobic drug molecules (such as one or more of the compounds described herein) can permeate biological barriers while maintaining a lipophilic compound in aqueous solution. Release of the drug molecule (e.g., one or more of the compounds described above) (through cleavage of hydrogen or ionic bonds between the cyclodextrin and the guest molecule) can be achieved by controlled degradation of the complex due to pH change or enzymatic action. Thus, one or more of the above-described compounds can be inserted into the torus of a cyclodextrin in general, and a β-cyclodextrin in particular.

As noted above, topical, systemic, intraocular, and periocular (including subconjunctival, sub-Tenon's, and/or retrobulbar) administration are contemplated. Furthermore, intravitreal administration is contemplated, wherein sustained-release devices or implants can be employed in connection with intravitreal administration of one or more of the above-described compounds. Still further, microspheres and liposomes can be employed in connection with intravitreal administration of one or more of the above-described compounds.

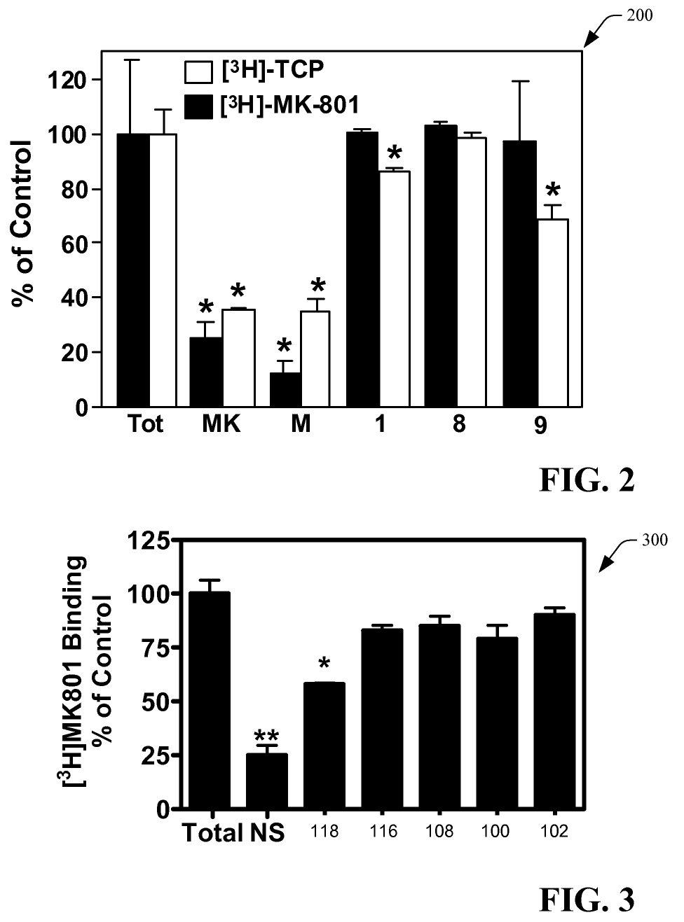

With reference now to FIG. 2, an example graph 200 illustrating data pertaining to NMDA channel block is depicted. One or more of the polycyclic compounds of FIGS. 1a-1l discussed above do not bind to the MK-801 binding site and only weakly to the TCP binding site at concentrations tested up to a maximum of 100 μM. Such data depicted in the graph 200 may suggest that the pentacyclo-undecylamines interact with a different binding site in the NMDA receptor/ion channel complex. This behavior may be related to structure of compounds, and for some compounds may be directed by ππ type aromatic stacking facilitated through a “complementary” aromatic amino acid located at the entrance of the NMDA channel pore. Such an interaction can allow the compounds described above to show fast “in-out” kinetics, as the side-chain can afford sufficient flexibility to the “cage” to move in and out of the channel pore while also anchoring it close to its site of action.

The graph 200 illustrates an interaction of the example compound 120 (FIG. 1l) (labeled 1 in the graph 200), and the following compound (8) and (9), respectively:

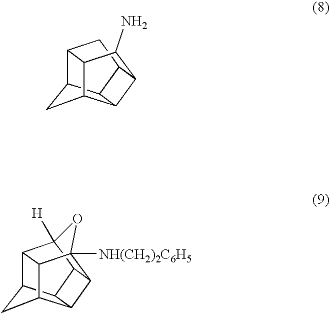

The values shown in the graph 200 are mean (pmol/mg protein) ±S.E.M. of a plurality of experiments. Abbreviations are: Total binding (Tot) or binding in the presence of cold MK-801 (MK), Memantine (M), and compounds (1), (8), and (9), each at 100 μM. Statistical significance compared to the total binding in a t-test, *P<0.05.

Now referring to FIG. 3, an example graph 300 that illustrates that at least a subset of the compounds described above have an affinity for the MK-801 binding site in the NMDA channel is presented. More particularly, some triquinylamine compounds may have an affinity for the MK-801 binding site in the NMDA channel. The triquinane-derived azatricycloundecane compounds 100, 102, 108, 116, and 118 (FIGS. 1a, 1b, 1e, 1i, and 1j, respectively) were tested for an ability to displaced labeled MK-801, and the results of such testing are shown in FIG. 3. The graph 300 also illustrates total binding of radiolabeled MK-801 (Total) and non-specific binding of unlabelled MK-801 (NS). The addition of NMDA (100 μM) and Gly (100 μM) are shown to have resulted in activation of the NMDA channel and (if competitive), displacement of labeled and unlabeled MK-801 compounds. Specific [³H]MK-801 binding to the homogenate can be estimated by subtracting non-specific binding (NS) obtained in the presence of 100 μM unlabeled MK-801 from the total binding. Specific binding represented 75% of the total binding and non-specific binding only 25% of total binding at 5 nM [³H]MK-80 1. Unlabeled MK-80 1 used to determine the non-specific binding also served as a reference compound. NMDA-stimulated displacement for each tested compound was calculated as the difference between the measured value for the labeled MK-801 in the presence of each test compound, and the total binding, although the purpose of the graph 300 represents only the non-specific binding for that particular compound. The compound 118 displaced [³H]MK-801 by 41.09±4.1%. For the compounds 116, 108, 100, and 102, respectively, there was no statistically significant displacement of [³H]MK-801, and displacement values were calculated to be 16.93±4.1%, 14.35±7.9%, 20.85±11.7%, and 9.28±6.17%, respectively. Statistical significance compared to total binding in a t-test is (*) p<0.05, (**) p<0.001.

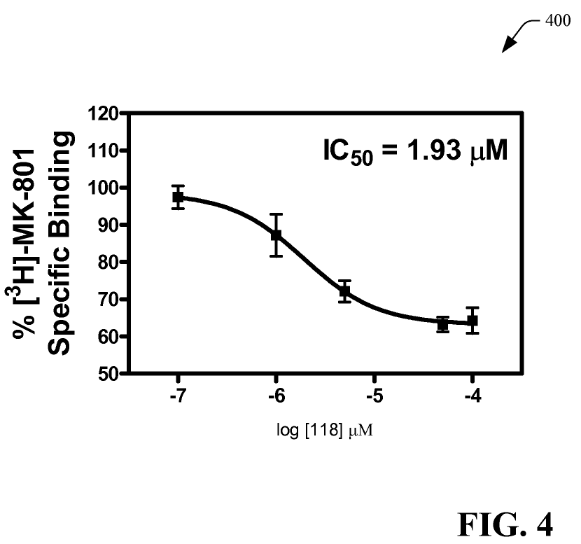

Referring now to FIG. 4, an example graph 400 that illustrates dose-dependent effect of compound 118 shown in FIG. 1j on [³H]MK-801 binding in the presence of NMDA (100 μM) and Gly (100 μM). More particularly, the graph 400 depicts results of a dose-response curve fitting that was performed to determine the IC₅₀ value for the compound 118. The IC₅₀ value for compound 118 was found to be 1.93±0.018 μM. Fit of the dose-response relationship to a sigmoidal curve was found to have an r² value of 0.9988. The Hill slope values for compounds with high-affinity as NMDA antagonists (MK-801, Memantine, and NGP1-01) are near unity, with lower potency compounds having greater Hill slop values. The Hill slope value for the compound 118 was −1.157±0.052.

Amongst the triquinane-derived compounds (compounds 100, 102, 108, 116, and 118), the benzylamine derivative (compound 118) was found to have the highest affinity for the NMDAR. Amongst the remaining compounds tested, an increase in chain length (compound 100) was found to lead to a slight increase in affinity for the NMDAR.

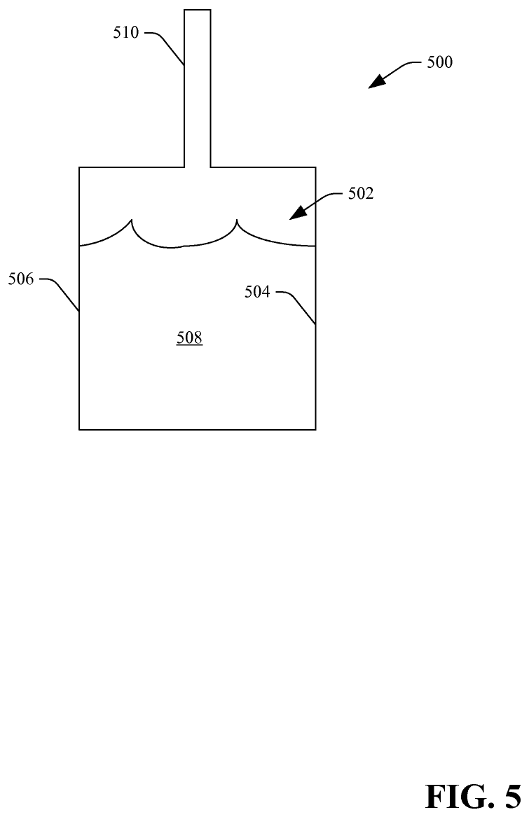

With reference to FIG. 5, an example apparatus 500 that can be used in applying an aqueous solution directly to an eye of an individual is illustrated. The apparatus 500 includes a reservoir 502, the reservoir 502 having at least one interior wall 504 and at least one exterior wall 506. The reservoir 502 is configured to retain an aqueous solution 508, wherein the aqueous solution includes at least one of the compounds shown and described above. In an example, the polycyclic compound may be caged polycyclic compounds. For instance, the caged polycyclic compound may be NGP1-01 and/or derivatives thereof. In another example, the caged polycyclic compound may be selected from one or more compounds disclosed in European Patent No. EP 0312245, which is incorporated in its entirety herein. Still further, the polycyclic compound may be any of the compounds of the general structures shown above, including the compounds 100, 102, 104, 106, 108, 110, 112, 114, 116, and/or 118.

The apparatus 500 additionally includes an outlet 510 that is configured to transmit a portion of the aqueous solution from the reservoir 502 to an exterior of an eye upon pressure being applied to the exterior wall 506 of the reservoir 502. Once applied to the eye, the polycyclic compound can facilitate prevention and/or treatment of retinal ganglion cell neurodegeneration as found in glaucomatous blindness and/or diabetic retinopathy. More specifically, at least the polycyclic compound of the aqueous solution 508 crosses through various layers of the eye and enters the ocular fluid of the eye. The polycyclic compound then acts as an L-type calcium channel blocker and also attenuates exitotoxicity by modulating calcium entry through an NMDA receptor in the eye. The polycyclic compound may additionally attenuate calcium release from intracellular stores, as described above. A multimodal compound has heretofore not been utilized for treatment of ocular neurodegenerative diseases. L-type calcium channels and the NMDA receptors are located on ocular neurons in the retina and ocular nerve in the eye. The blockage of the L-type calcium channels and modulation of calcium entry through NMDA receptors and/or calcium from intracellular stores prevents or reduces an episodic pathway that leads to neuronal death. Contrary to the conventional wisdom that increased intraocular pressure is the cause of glaucoma, in some instances, accumulation of calcium may be the cause of glaucoma and increased intraocular pressure may be a symptom.

While described above as being topically applied to the eye by way of, for example, an eye drop device, it is to be understood that other manners for treating ocular neurodegenerative diseases are contemplated and have been described above. Moreover, a different mechanism from that shown herein may be used to topically apply treatment directly to the eye.

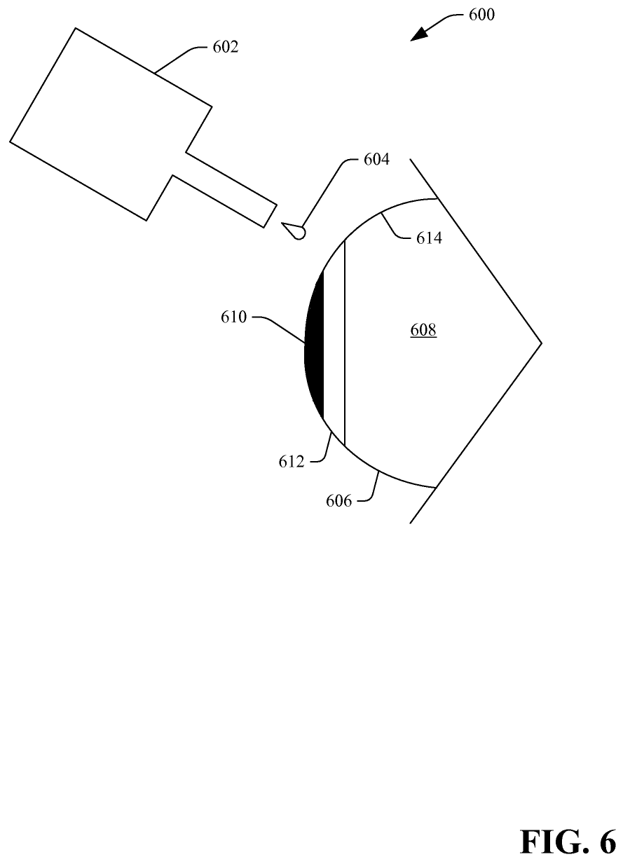

Referring now to FIG. 6, an illustration 600 of treatment of glaucoma or another ocular neurodegenerative disease is depicted. A container 602 that includes an aqueous solution is positioned to apply at least a drop 604 of the aqueous solution to a surface 606 of an eye 608. The aqueous solution, as noted above, includes a polycyclic compound, such as one or more of the compounds of the general formulas presented above. In one example, the drop 604 may be applied on the cornea of the eye 608 near a pupil 610 of the eye 608. In another example, the drop 604 may be applied near an iris 612 of the eye 608. In yet another example, the drop 604 may be applied to avoid the pupil 610 and/or the iris 612.

As noted above, at least the polycyclic compound traverses the layers of the eye 608 (e.g., the cornea and other layers) and enters the ocular fluid of the eye 608. The polycyclic compound reaches ocular neurons in the retina of the eye 608 and acts as an L-type calcium channel blocker with respect to such neurons as well as a calcium blocker for NMDA receptors. The polycyclic compound additionally reaches the optic nerve of the eye and neurons therein and acts as an L-type calcium channel blocker with respect to such neurons as well as a calcium blocker for NMDA receptors.

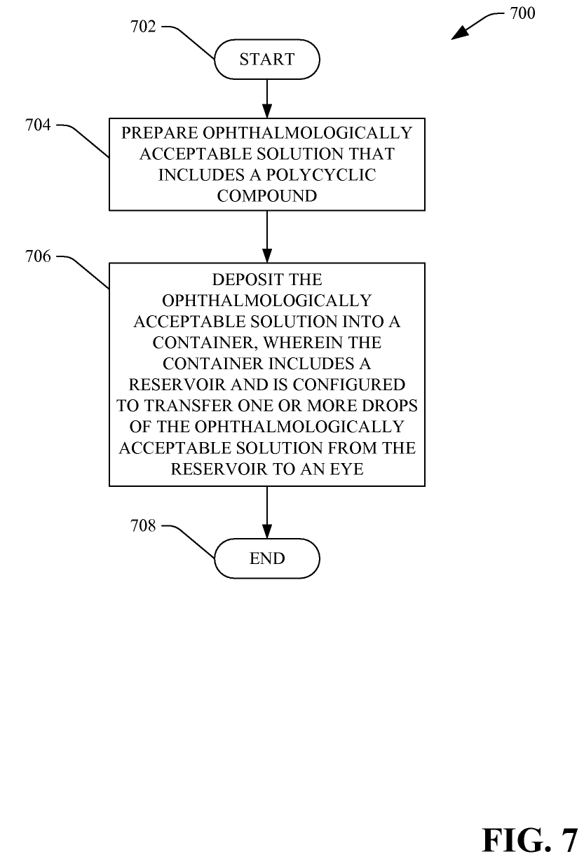

With reference now to FIGS. 7-9, various example methodologies are illustrated and described. While the methodologies are described as being a series of acts that are performed in a sequence, it is to be understood that the methodologies are not limited by the order of the sequence. For instance, some acts may occur in a different order than what is described herein. In addition, an act may occur concurrently with another act. Furthermore, in some instances, not all acts may be required to implement a methodology described herein.

Referring specifically to FIG. 7, a methodology 700 for treating an ocular neurodegenerative disease, such as glaucoma or diabetic retinopathy, is illustrated. The methodology 700 starts at 702, and at 704 an aqueous solution that includes a polycyclic compound is prepared. At 706, the aqueous solution is deposited into a container that is suitable, for example, for sale to consumers. The container includes a reservoir that retains the aqueous solution and is configured to transfer one or more drops of the aqueous solution from the reservoir to an eye, wherein the eye has been diagnosed with a neurodegenerative disease such as glaucoma or diabetic retinopathy. While described as being an aqueous solution, it is understood that the method can be modified such that an ingestible tablet is produced for treatment of an ocular neurodegenerative disease, such as glaucoma. The methodology 700 completes at 708.

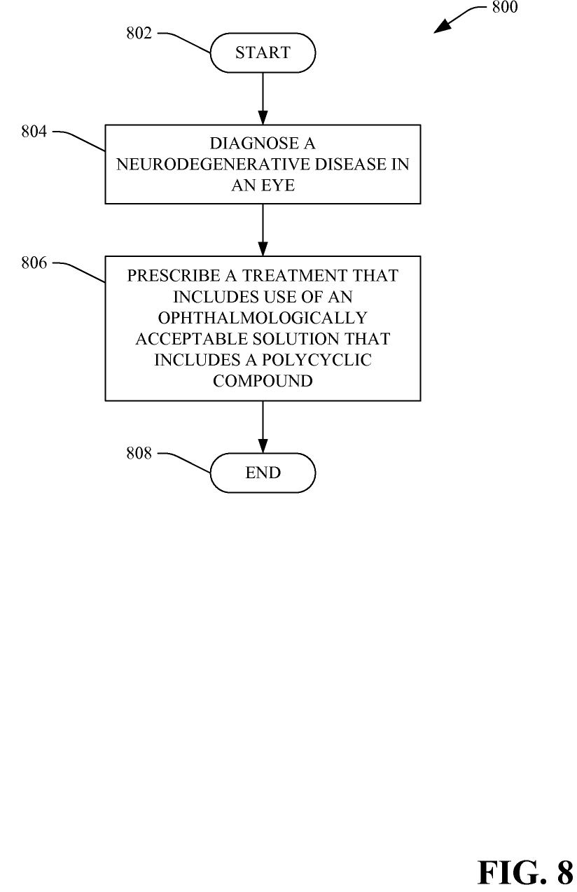

Turning now to FIG. 8, a methodology 800 for prescribing treatment for an ocular neurodegenerative disease, such as glaucoma, is illustrated. The methodology 800 starts at 802, and at 804 a neurodegenerative disease in an eye is diagnosed. For example, the diagnosis may be made by an optometrist or other expert in a field relating to the eye. At 806, treatment for the neurodegenerative disease is prescribed, wherein the prescribed treatment includes use of an aqueous solution that includes a polycyclic compound, such as a polycyclic compound of at least one of the general formulas described above. The methodology 800 then completes at 808.

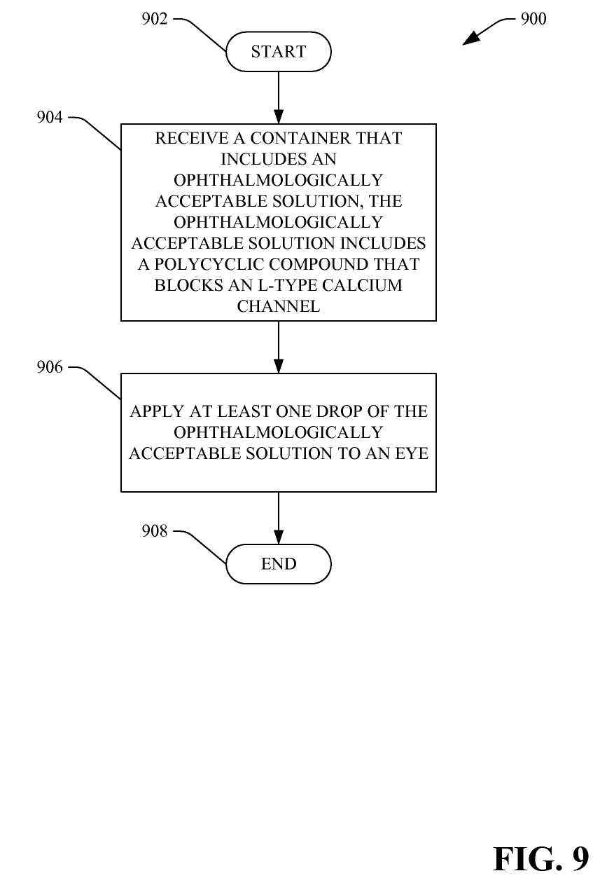

Referring now to FIG. 9, a methodology 900 for application of an aqueous solution to an eye is illustrated, wherein the eye suffers from a neurodegenerative disease and aqueous solution includes a polycyclic compound that at least acts as an L-type calcium channel blocker and a calcium blocker for NMDA receptors. The polycyclic compound may also act to attenuate calcium release from intracellular stores. The methodology 900 starts at 902, and at 904 a container is received that includes an aqueous solution. The aqueous solution includes a polycyclic compound that at least blocks an L-type calcium channel and modulates calcium entry through an NMDA receptor channel. At 906, at least one drop of the aqueous solution is applied to an eye, wherein the eye suffers from a neurodegenerative disease such as glaucoma. The methodology 900 ends at 908.

It is noted that several examples have been provided for purposes of explanation. These examples are not to be construed as limiting the hereto-appended claims. Additionally, it may be recognized that the examples provided herein may be permutated while still falling under the scope of the claims.

What is claimed is:

1. An azatricyclo[6.3.0.0^{2,6}] undecane compound suitable for treating an ocular neurodegenerative disease of the following general formula: 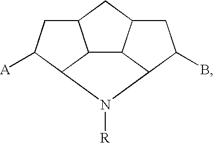
wherein R includes one of an alkyl group that has one to twelve carbon atoms or a phenyl group.

2. The compound of claim 1, wherein R includes the alkyl group.

3. The compound of claim 2, the alkyl group is a linear alkyl group.

4. The compound of claim 2, wherein the alkyl group is a branched alkyl group.

5. The compound of claim 1, wherein R includes the phenyl group.

6. The compound of claim 5, wherein the phenyl group includes a linear, cyclic, or branched alkyl group.

7. The compound of claim 6, wherein the linear, cyclic, or branched alkyl group includes one to twelve carbon atoms.

8. The compound of claim 6, wherein the linear, cyclic, or branched alkyl group includes a hydroxyl substituent.

9. The compound of claim 6, wherein the linear, cyclic, or branched alkyl group includes a halogen substituent.

10. The compound of claim 1, wherein R is one of —CH₃, —(CH₂)₃—CH₃, —(CH₂)₇—CH₃, —CH₂—C₆H₅, —(CH₂)₂—C₆H₅, —(CH₂)₃—C₆H₅, —CH₂CH₃, —(CH₂)₁₁—CH₃, —CH₂CH(CH₃)₃, —(CH₂)₅—CH₃, —C(CH₃)₂—CH₂—C(CH₃)₃, —(CH₂)₉—CH₃, —CH₂—CH₂—OH, or —C₆H₅.

11. The compound of claim 1, wherein one of A or B is hydrogen.

12. The compound of claim 11, wherein the other of A or B is a methyl group.

13. The compound of claim 1, wherein A and B are hydrogen.

14. The compound of claim 1, wherein the ocular neurodegenerative disease is one of glaucoma or diabetic retinopathy.

15. The compound of claim 1 being N-phenylpropyl-3,11-azatricyclo[6.3.0.0^{2,6}] undecane.

16. The compound of claim 1 being N-(pyridin-4-ylmethyl)-3,11-azatricyclo[6.3.0.0^{2,6}] undecane.

17. The compound of claim 1 being N-(3-methoxybenzyl)-3,11-azatricyclo[6.3.0.0^{2,6}] undecane.

18. The compound of claim 1 being N-cyclohexylmethyl-3,11-azatricyclo [6.3.0.0^{2,6}] undecane.

19. The compound of claim 1 being N-benzyl-3,11-azatricyclo [6.3.0.0^{2,6}]undecane.

20. A pharmaceutical composition comprising, as an active ingredient, the compound of claim 1 in combination with a diluent to create an aqueous solution that is configured for topical administration for treatment of an ocular neurodegenerative disease.
